SEQUENCE LISTING (1 sequence)

SEQ ID NO: 1 - protein, 102 aa, synthetic construct
MYGKLNEILEQFDEVLDQLDKNWHKRGNNLHDIEDELHQLVKHFHHFMQGHKNEGKLQDMFDQMQQLLENFDNHLQKRNETVHHIHEKLNQLIHQFDHLVHR

(19) United States
(12) Patent Application Publication
(10) Pub. No.: US 2026/0275188 A1
(43) Pub. Date: Sep. 17, 2026

(54) METHOD FOR PRODUCING SEMICONDUCTOR QUANTUM DOTS

(71) Applicant: The Trustees of Princeton University, Princeton, NJ (US)

(72) Inventors: Michael Hecht, Skillman, NJ (US); Sarangan Chari, Princeton Junction, NJ (US); Leah Spangler, Monmouth Junction, NJ (US)

(73) Assignee: The Trustees of Princeton University, Princeton, NJ (US)

(21) Appl. No.: 19/695,115

(22) Filed: Jun. 2, 2026

Related U.S. Application Data
(62) Division of application No. 18/179,659, filed on Mar. 7, 2023, now Pat. No. 12,668,740.
(60) Provisional application No. 63/317,120, filed on Mar. 7, 2022.

(51) Int. Cl. C09K 11/56; B01J 20/20; B01J 31/06; B82Y 20/00; B82Y 40/00; C01G 11/02; C07K 14/00; C30B 7/00; C30B 29/46; C30B 29/48; C30B 29/50

(52) U.S. Cl. CPC C09K 11/565; B01J 20/205; B01J 31/062; C01G 11/02; C07K 14/001; C30B 7/00; C30B 29/46; C30B 29/48; C30B 29/50; B01J 2231/005; B01J 2531/002; B82Y 20/00; B82Y 40/00; C01P 2004/64; C01P 2006/60

(57) ABSTRACT

A catalyzing agent is provided that includes a de novo protein bound to a pyridoxal phosphate (PLP), wherein the de novo protein comprises an amino acid sequence matching a binary pattern of polar (P) and nonpolar (N) amino acids, and wherein the de novo protein is a PLP binding de novo Protein. A system is provided for producing a semiconductor nanocrystal that includes a buffer solution containing a metal salt, a sulfur-containing substrate in the buffer solution, and a de novo protein in the buffer solution, wherein the de novo protein comprises an amino acid sequence matching a binary pattern of polar (P) and nonpolar (N) amino acids, and wherein the de novo protein is a PLP binding de novo Protein.

### CROSS-REFERENCE TO RELATED APPLICATIONS

This application is a divisional under 35 U.S.C. § 121 of and claims priority under 35 U.S.C. § 120 to U.S. patent application Ser. No. 18/179,659, filed Mar. 7, 2023, entitled “Method for Producing Semiconductor Quantum Dots”, which in turn claims benefit of U.S. Provisional Patent Application No. 63/317,120, filed Mar. 7, 2022.

### STATEMENT REGARDING FEDERALLY SPONSORED RESEARCH OR DEVELOPMENT

This invention was made with government support under Grant No. MCB-1947720 awarded by the National Science Foundation. The government has certain rights in the invention.

### SEQUENCE LISTING

The instant application contains a Sequence Listing which has been submitted electronically in XML file format and is hereby incorporated by reference in its entirety. Said XML copy, created on Jun. 23, 2023, is named Princeton-83403_SL.xml and is 2,056 bytes in size.

### TECHNICAL FIELD

The present disclosure is drawn to techniques for producing semiconductor quantum dots, and specifically to using de novo proteins for producing quantum dots.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

There are numerous applications of quantum dots in a biological context. However, there are few methods for producing bio-compatible quantum dots as most traditional synthesis methods use toxic-precursors which are difficult or impossible to remove.

### BRIEF SUMMARY

Various deficiencies in the prior art are addressed below by the disclosed compositions of matter and techniques.

In various aspects, a method for producing a semiconductor nanocrystal may be provided.

The method may include producing and/or purifying the de novo protein prior to introducing the protein into the buffer. The de novo protein may include an amino acid sequence matching a binary pattern of polar (P) and nonpolar (N) amino acids, and the de novo protein may be a pyridoxal phosphate (PLP) binding de novo Protein. In some embodiments, the de novo protein may include an amino acid sequence having a sequence having at least 80% sequence identity to MYGKLNEILE QFDEVLDQLD KNWHKRGNNL HDIEDELHQL VKHFHHFMQG HKNEGKLQDM FDQMQQLLEN FDNHLQKRNE TVHHIHEKLN QLIHQFDHLV HR [SEQ ID NO. 1]. In some embodiments, the buffer may include, e.g., N-Methylmorpholine (NMM).

The method may include combining various components in a buffer, including a de novo protein as disclosed herein, a metal salt, a sulfur-containing substrate, and PLP to the buffer. The metal salt may comprise, e.g., Cd. The sulfur-containing substrate may be an amino acid, and may be, e.g., L-cysteine and/or D-cysteine.

The method may include allowing a semiconductor nanocrystal to form. This may include allowing the de novo protein to bind to a PLP cofactor to generate catalytic activity, and using the catalytic activity to produce the semiconductor nanocrystal. The semiconductor nanocrystal may be a metal chalcogenide semiconductor nanocrystal, such as a CdS nanocrystal. In some embodiments, the semiconductor may include a single crystal phase.

The method may include allowing the formed semiconductor nanocrystal to grow to a predetermined size.

The method may include capturing H₂S generated during the formation of the semiconductor nanocrystal, and may include using the captured H₂S to synthesize a different material (such as a reduction of graphene oxide).

In various aspects, a catalyzing agent may be provided. The catalyzing agent may include a de novo protein as disclosed herein, bound to a pyridoxal phosphate (PLP) cofactor.

In various aspects, a system may be provided. The system may include a buffer solution containing a metal salt, a sulfur-containing substrate in the buffer solution, and a de novo protein as disclosed herein in the buffer solution.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart of an embodiment of a method.

FIGS. 2A and 2B are graphs showing the time course of absorbance (2A) and fluorescence spectra (2B) of CdS nanocrystal solutions grown with 0.25 mg/mL ConK and 32 mM PLP.

FIG. 3 is a graph showing the required time for the onset of CdS nanocrystal formation based on the concentrations of a de novo protein and PLP.

FIG. 4 is a graph showing average H2S generation over time for a solution containing 2.5 mM L-cysteine, 0.25 mg/mL ConK, and either 40 or 80 μM PLP.

FIG. 5 is a graph showing CD spectra of chiral CdS quantum dots synthesized using L- or D-cysteine; L-cysteine and D-cysteine alone give no signal in the excitonic region (>250 nm).

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Rather than identifying a natural catalyst, it has been determined that a de novo protein may be used to catalyze the mineralization of semiconductor quantum dots, such as CdS nanocrystals.

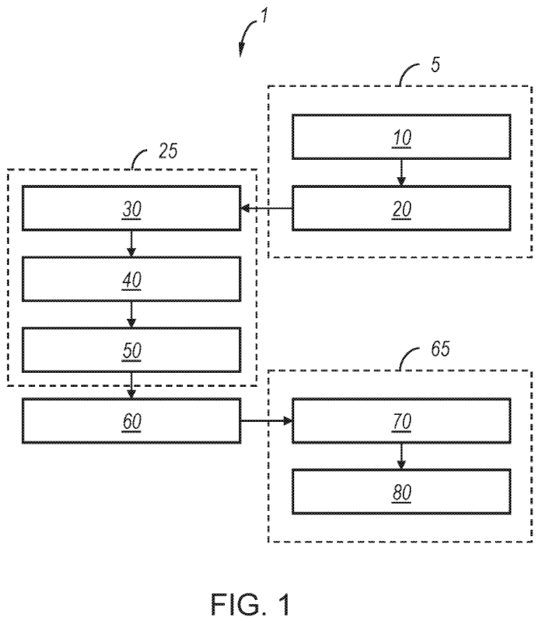

Disclosed herein is a biological method for producing semiconductor quantum dots using de novo proteins. Referring to FIG. 1, the method 1 may some or all of the following steps.

First, the method may include one or more steps related to preparing 5 a de novo protein for use in the quantum dot synthesis. This may include producing 10 the de novo protein. De novo proteins are created in the laboratory from amino acid sequences that share no common ancestry with naturally evolved systems. One class of de novo proteins, pioneered by the Hecht group comprises large combinatorial collections of sequences that are designed using a strategy that relies on a binary code for protein design. Such proteins, including techniques for creating them, are well known. See, e.g., Kamtekar, S., et al., “Protein design by binary patterning of polar and nonpolar amino acids”, Science 262, 1680-1685 (1993) and Hecht, M. H., et al., “De novo proteins from designed combinatorial libraries”, Protein Sci. 13, 1711-1723 (2004), the contents of which are each incorporated by reference herein in its entirety. The binary code specifies the pattern of polar and nonpolar amino acids while allowing the exact identities of the individual side chains at each position to vary combinatorially. This approach facilitates the construction of libraries of proteins containing millions of different sequences. In particular, the binary code has produced several libraries of proteins that fold into 4-helix bundle structures. Importantly, these proteins were neither selected by nature nor do they have any relationship to natural biochemical pathways.

The de novo protein should be a PLP Binding de novo Protein. As used herein, the term “PLP Binding de novo Protein” refers to a polypeptide comprising or consisting of four α-helices, wherein each α-helix comprises a binary patterned sequence of seven amino acid residues, or heptad sequence, defined by [PNPPNNP]ₙ, where each “P” is independently selected from the polar amino acid residues Lys (K), His (H), Glu (E), Gln (Q), Asp (D), Asn (N), Thr (T) and Ser (S), each “N” is independently selected from the nonpolar amino acid residues Phe (F), Leu (L), He (I), Met (M), Val (V) and Trp (W), and n is an integer from 2 to 6, and preferably n=3 or 4.

The heptad sequences in an α-helix containing more than one heptad sequence can be identical (i.e., repeats of the same heptad sequence) or they can be different (i.e., each PNPPNNP heptad sequence within the same α-helix can have a different amino acid sequence). Furthermore, the amino acid composition of the α-helices in a PLP Binding de novo Protein can vary from helix to helix such that, for example, each of the α-helices in the protein will have a different amino acid sequence.

A PLP Binding de novo Protein may optionally include additional amino acid residues, for example, N-terminal to the first α-helix and/or C-terminal to the last α-helix in the protein.

A PLP Binding de novo Protein will also include interhelical turns between the α-helices, wherein each interhelical turn includes one or more amino acid residues (preferably between 2-8 residues, and more preferably 4-6 residues), selected from amino acids that are compatible with the formation of turns. Such amino acids may include, e.g., Gly (G), His (H), Gin (Q), Asn (N), Asp (D), Glu (E) and Lys (K)).

The PLP Binding de novo Protein is generally between 75 amino acid residues and 160 amino acid residues in length (e.g., 102 amino acid residues). In some embodiments, the PLP Binding de novo Protein is between 96 and 102 amino acid resides in length.

In some embodiments, the de novo protein may include an amino acid sequence having a sequence having at least 80%, at least 90%, at least 95%, at least 97%, at least 98%, at least 99%, or 100% sequence identity to the sequence of the ConK protein: MYGKLNEILE QFDEVLDQLD KNWHKRGNNL HDIEDELHQL VKHFHHFMQG HKNEGKLQDM FDQMQQLLEN FDNHLQKRNE TVHHIHEKLN QLIHQFDHLV HR [SEQ ID NO. 1].

The preparation steps may include purifying 20 the protein. This may be done in any appropriate purification technique. For example, in some embodiments, the protein includes an affinity tag, and/or purification may utilize, e.g., affinity chromatography.

### Example 1 (Expression and Purification of ConK and Mutants)

ConK and a mutated protein, K56R, were expressed using recombinant E. coli as described previously. See K. J. Hoegler, K. J. and Hecht, M. H., “A de novo protein confers copper resistance in Escherichia coli: Novel protein confers copper resistance”, Protein Sci. 25, 1249-1259 (2016). The plasmid encoding ConK was transformed into BL21 (DE3) E. coli. Following transformation, cells were plated onto LB supplemented with chloramphenicol (30 μg/mL) and grown overnight at 37° C. A single colony was then used to inoculate a 2 L volume of LB broth supplemented with 30 μg/mL chloramphenicol. This culture was grown at 37° C. until the cell concentration reached an OD₆₀₀ of about 0.5. At this time, IPTG (final concentration of 100 M) was added to induce protein expression, followed by incubation at 18° C. overnight. Following overexpression, cells were recovered by centrifugation at 3,000×g and frozen at −20° C. for later use.

Immediately prior to purification, frozen cell pellets containing overexpressed ConK were thawed and resuspended in 25 mL of 50 mM Tris and 300 mM NaCl (buffer A) at 4° C. for 30 min. The resuspended cells were then sonicated on ice using a probe-tip sonicator for a total of 4 min (pulse 10/50 s on/off) and 30% amplitude. Next, the lysate was clarified by centrifugation at 35,000×g for 30 min to separate the soluble overexpressed proteins from cell debris. The protein-rich supernatant was filtered using 0.22-μM PES membrane syringe filters.

The proteins were purified using immobilized metal affinity chromatography (IMAC) followed by size-exclusion chromatography (SEC). While ConK does not carry a canonical histidine tag (6× His) typically required for IMAC, its sequence contains a high percentage of histidine (ca. 14%) enabling nickel binding. First, the filtered supernatant containing ConK was applied to a 5-mL HisTRAP column (GE Healthcare) and equilibrated with five column volumes of running buffer containing 50 mM Tris and 300 mM sodium chloride at pH 7.5. A second wash step using five column volumes of 50 mM imidazole, 50 mM Tris, and 300 mM NaCl at pH 8 removed any nonspecifically bound proteins. ConK was then eluted using 375 mM imidazole, 50 mM Tris, and 300 mM NaCl. The eluates were analyzed by SDS-PAGE, and the appropriate fractions were pooled and further purified by SEC on a HiLoad Superdex 75 26/600 column (GE Healthcare). SEC also removes imidazole from the IMAC stage. This two-stage process typically yields proteins of greater than 95% purity, as assessed by SDS-PAGE. ESI-MS measurements were performed on 10-uL of protein following separation on an HPLC (Agilent 6220 accurate-mass time-of-flight LC/MS). Further characterization of ConK oligomerization was performed using analytical ultracentrifugation (Optima AUC from Beckman Coulter Life Sciences with absorbance and interference detection modules).

The method may include combining 25 various components to a buffer, e.g., to form a solution. This may include adding 30 a de novo protein as disclosed herein to the buffer.

Any appropriate buffer may be utilized. For example, in some embodiments, the buffer may include a buffering agent such as, e.g., N-Methylmorpholine (NMM), a salt (such as NaCl), 4-(2-hydroxyethyl)-1-piperazineethanesulfonic acid (HEPES), and/or tris(hydroxymethyl)aminomethane (Tris). In some embodiments, the buffering agent may be present in a concentration of 100 mM or less. In some embodiments, the buffering agent may be present in a concentration of 50 mM or less. The buffer may be an aqueous buffer. The pH value of the buffer may be adjusted to a pH value of 6 to 8.5.

The method may include adding 40 a metal salt and a sulfur-containing substrate to the solution. The method will generally include the desulfurization of the sulfur-containing substrate, catalyzed by the de novo protein, to form, e.g., H₂S, which can then be used to react with the metal salt to form a semiconductor quantum dot.

In some embodiments, the metal salt may be selected from I-VI, II-VI, IV-VI and III-V semiconductor metals. The metal salt may comprise Cd, Ce, Cu, Fe, Hg, In, Ga and/or Zn. In some embodiments, the metal salt may comprise a Cd metal. In some embodiments, the metal salt may be, e.g., CdCl₂.

The metal salt may be present in any appropriate amount. In some embodiments, the concentration of the metal salt in the solution is 5 mM or less. In some embodiments, the concentration is 1 mM or less.

The sulfur-containing substrate may be an amino acid, and may be, e.g., L-cysteine and/or D-cysteine.

In some the sulfur-containing substrate may be present in any appropriate amount. In some embodiments, the concentration of the sulfur-containing substrate in the solution is 20 mM or less. In some embodiments, the concentration is 10 mM or less.

The method may include adding 50 pyridoxal phosphate (PLP) cofactor to the buffer. In some embodiments, the PLP may be added at a concentration of 0-200 μM. In some embodiments, the PLP may be added at a concentration of 0-80 μM. The PLP may form a Schiff base, e.g., with a lysine in the de novo protein.

It will be understood that the various addition steps may be performed in any order.

The method may include allowing 60 a semiconductor nanocrystal to form. This may include allowing the de novo protein to bind to a PLP cofactor to generate catalytic activity, and using the catalytic activity to produce the semiconductor nanocrystal.

The resulting semiconductor nanocrystal will depend upon the components in the reaction mixture. If the metal salt comprises Cd, for example, the semiconductor nanocrystal may be a metal chalcogenide semiconductor nanocrystal, such as a CdS nanocrystal.

### Example 2. (Synthesis of Quantum Dots)

Various quantum dot syntheses were initiated by first preparing a solution of 1 mM cadmium chloride (99%, from Sigma-Aldrich) and 10 mM L-cysteine (>97%, from Sigma-Aldrich) in 50 mM NMM at pH 7.5. To this solution, various concentrations (0 to 0.25 mg/mL) of ConK were added, followed by various amounts of PLP (0 to 80 μM), to initiate the biomineralization process. Following addition of PLP, the reaction mixture was placed in a 37° C. incubator with shaking for up to 48 hrs.

The formed semiconductor nanocrystal may be allowed to grow to a predetermined size. This may be done by adjusting the amount of time the reaction mixture is allowed to incubate. A difference in size can be seen via, e.g., a change in the fluorescence of quantum dot solutions over time.

### Example 3. (Controlling Size of Nanocrystals)

As discussed herein, the de novo protein may be used to catalyze the desulfurization of cysteine to H₂S, which can be used to synthesize nanocrystals in solution (in this example, synthesizing CdS nanocrystals).

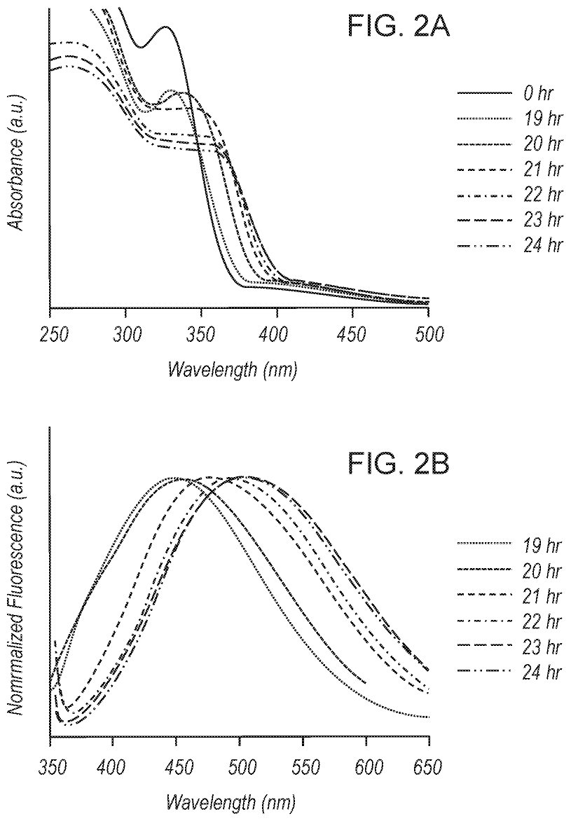

A solution containing CdCl₂, cysteine, ConK (0.2 mg/mL), and PLP (32 mM) was incubated. Absorbance and fluorescence were monitored for 24 hours. At early times, the only observable peak was at 330 nm from the expected cysteine-PLP complex. However, after incubation for 19 h at 37° C., this peak decreased, with a concomitant increase of a peak at 340 nm, indicating the formation of CdS nanocrystals. See FIG. 2A.

Following their initial appearance, the CdS nanocrystals continued to grow for approximately 8 h, as shown by an absorbance peak that moved to longer wavelengths over time. See FIG. 2A. After 24 h, the peak stabilized at 380 nm, suggesting the nanocrystals grew to a fixed final size. The wavelength of the absorbance can be correlated to the size of the nanocrystals using a size-dependent Beer law calculation. Based on the absorbance maximum, the CdS nanocrystals appear to shift in size from approximately 1.47 to 2.5 nm over the course of 24 h. The fluorescence peak maxima of the CdS nanocrystal populations, shown in FIG. 2B, also shows a shifting wavelength over time. The size distribution of the resulting CdS nanocrystal populations can be evaluated by calculating the full width at half maximum (FWHM) of the fluorescence peak, which indicates population dispersity. The FWHM values, seen in Table 1, below, range from 159 to 177 nm, consistent with relatively broad size distributions commonly observed for CdS nanocrystals synthesized via biomineralization.

TABLE 1
Calculated FWGM values for the fluorescence spectra in FIG. 2B.

| Growth Time (hours) | FWHM (nm) |
|---|---|
| 19 | 156 |
| 20 | 170 |
| 21 | 169 |
| 22 | 163 |
| 23 | 177 |
| 24 | 171 |

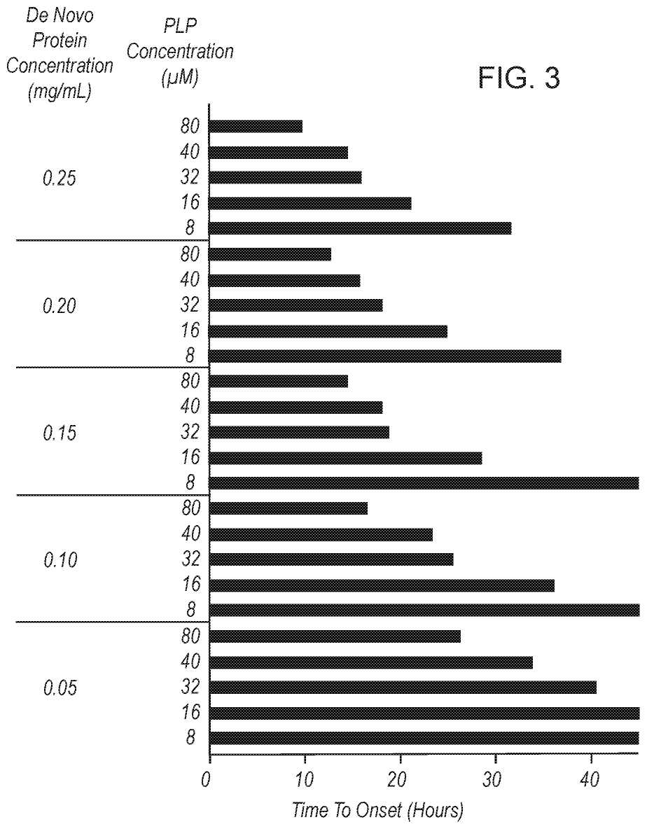

Not surprisingly, the time required to reach the onset of CdS nanocrystal formation depended on the concentration of ConK and PLP. See FIG. 3. When higher concentrations of ConK (0.25 mg/mL) and PLP (80 μM) were used, the appearance of a peak at 340 nm occurred after only 10 h. For low concentrations of PLP and ConK (e.g., combinations of 8 to 16 mM PLP and 0.05 to 0.1 mg/mL ConK), the onset of CdS quantum dots was not observed during the experimental time frame of 48 h. The long dwell time required for the formation of quantum dots suggests a slow generation rate of H₂S by ConK, which is not surprising for a de novo protein that was not explicitly designed for this function.

The crystal morphology and nanocrystal size distribution were verified using XRD and TEM measurements. Importantly, semiconductor nanocrystals synthesized using the disclosed de novo proteins demonstrate improved stability compared to those obtained using naturally derived biomineralization pathways, making de novo biomineralization ideal for commercial implementation.

In some embodiments, the semiconductor may include a single crystal phase. This is a major difference between CdS quantum dots grown using the de novo proteins versus natural systems such as cystathionine γ-lyase (CSE). A mixed distribution of crystal phases is commonly observed for CdS nanocrystals synthesized at room temperature via natural biomineralization routes. In contrast, for synthesis of, e.g., CdS using the de novo proteins, a single phase of CdS, zinc blende, was observed. The dominance of zinc blende CdS is striking and indicates that the slow growth using ConK favors more controlled crystallization. This suggests that ConK could produce higher-quality crystallites at room temperature than those previously achieved by natural proteins.

To study the active site of one particular de novo protein, ConK, in the absence of an experimentally determined structure, AlphaFold was used to predict the structure. Because ConK appears to oligomerize in solution, the structure was modeled as either a monomer or a dimer. In the AlphaFold prediction, Lys56 (believed to be the active site for ConK) occurs in a semiexposed pocket in the 2nd interhelical turn of the protein. Because of the location of this turn, this binding pocket is the same in both the monomeric and dimeric states. Three relevant proximally close amino acids, His43, His46, and Phe47, were also identified. Aromatic amino acids are often observed in PLP-binding sites because they stabilize the pyridine ring through π-stacking. Similarly, histidines have also been shown to stabilize the pyridine ring by forming an H-bond with nitrogen. The proximity of both Phe and His side chains is consistent with natural PLP-dependent enzyme active sites. It is noted that although the active site of ConK shares several features with the PLP-binding sites in CSE, such as forming a Schiff base with lysine and the presence of aromatic amino acids, the sequence and structure of the active site pocket are dramatically different. This is expected as ConK has no evolutionary ancestry and was isolated from a library of semirandom de novo sequences.

To assess the stereoselectivity of the ConK catalytic site, the L-cysteine was replaced with D-cysteine and the formation of CdS nanocrystals was monitored. Using D-cysteine led to an absorbance peak corresponding to CdS quantum dots, indicating that D-cysteine can also be turned over to H₂S by ConK. Notably, cysteine plays a dual role in the system, acting as both a reactant and a capping ligand that stabilizes the quantum dot surface. It was found that the D-isomer can also serve as a capping agent. Following filtration using a 10-kDa MWCO filter to remove cysteamine adducts, the resulting CdS quantum dot solutions were assayed for chirality by CD spectroscopy. The signal was found to be exactly opposite when using D-cysteine (see FIG. 5), indicating that D-cysteine acts as a mirror image capping ligand on the surface of the CdS quantum dot.

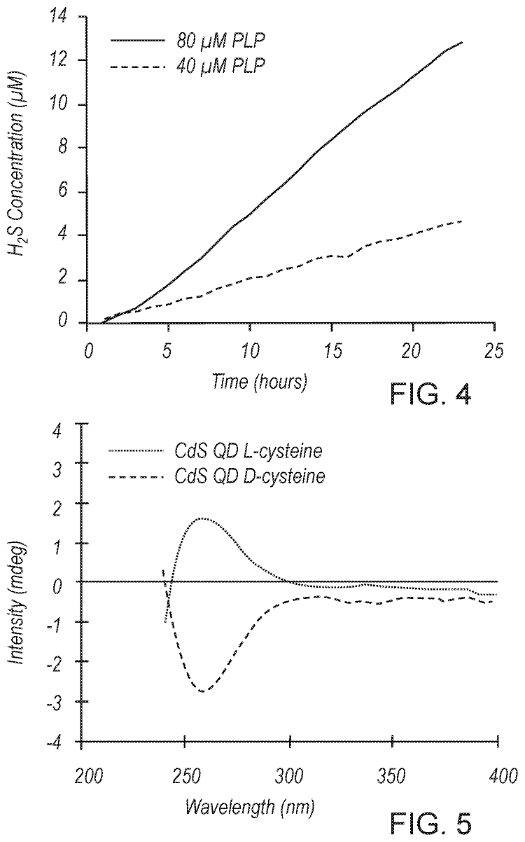

The method may include one or more steps related to managing 65 the byproducts of the synthesis reaction. This may include capturing 70 H₂S generated during the formation of the semiconductor nanocrystal (see, e.g., FIG. 4).

The captured H₂S may then be utilize for various purposes. For example, in some embodiments, the method may include using 80 the captured H₂S to synthesize a different material (such as a reduction of graphene oxide). Those of skill in the art will recognize the numerous reactions for which the captured H₂S could be utilized.

In various aspects, a catalyzing agent may be provided. The catalyzing agent may include a de novo protein as disclosed herein, bound to a pyridoxal phosphate (PLP) cofactor. In some embodiments, the PLP will be bound to a lysine of the de novo protein. As discussed previously, preferably, the lysine will be within a binding pocket that is proximally close to at least one histidine and/or at least one phenylalanine when folded. The term “proximally close” as used herein refers to being sufficiently close to stabilize the pyridine ring of the PLP, e.g., via engaging in π-stacking or forming an H-bond with nitrogen.

In various aspects, a system may be provided. The system may include a buffer solution containing a metal salt, a sulfur-containing substrate in the buffer solution, and a de novo protein as disclosed herein in the buffer solution. In some embodiments, the system may also include PLP.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed:

1. A catalyzing agent, comprising:
a de novo protein bound to a pyridoxal phosphate (PLP),
wherein the de novo protein comprises an amino acid sequence matching a binary pattern of polar (P) and nonpolar (N) amino acids, wherein the de novo protein is a PLP binding de novo Protein.

2. The catalyzing agent of claim 1, wherein the PLP is bound to a lysine of the de novo protein.

3. The catalyzing agent of claim 2, wherein the lysine is within a binding pocket that is proximally close to at least one histidine and/or at least one phenylalanine when folded.

4. A system for producing a semiconductor nanocrystal, comprising:
a buffer solution containing a metal salt;
a sulfur-containing substrate in the buffer solution; and
a de novo protein in the buffer solution;
wherein the de novo protein comprises an amino acid sequence matching a binary pattern of polar (P) and nonpolar (N) amino acids, wherein the de novo protein is a pyridoxal phosphate (PLP) binding de novo Protein.

5. The system of claim 4, wherein the system further comprises a PLP bound to the de novo protein.

6. The system of claim 5, wherein the PLP is bound to a lysine of the de novo protein.

7. The system of claim 6, wherein the lysine is within a binding pocket that is proximally close to at least one histidine and/or at least one phenylalanine when folded.
